(19) United States
(12) Patent Application Publication
(10) Pub. No.: US 2025/0135135 A1
(43) Pub. Date: May 1, 2025

(54) POSITIVE PRESSURE BREATHING CIRCUIT

(71) Applicant: Fisher & Paykel Healthcare Limited, Auckland (NZ)

(72) Inventor: David John Love, Auckland (NZ)

(21) Appl. No.: 18/837,123

(22) PCT Filed: Feb. 11, 2022
(86) PCT No.: PCT/NZ2022/050018

§ 371 Date: Aug. 8, 2024

(51) Int. Cl. A61M 16/00; A61M 16/08; A61M 16/20

(52) U.S. Cl. CPC A61M 16/024; A61M 16/0003; A61M 16/0883; A61M 16/209; A61M 2016/0027; A61M 2016/003; A61M 2202/0007; A61M 2202/0208; A61M 2202/0241; A61M 2202/025; A61M 2205/3303; A61M 2205/3327; A61M 2205/3334; A61M 2206/10; A61M 2230/005; A61M 2230/40; A61M 2240/00

(57) ABSTRACT

The present disclosure relates to a positive pressure breathing circuit and a method for ventilating a patient. The breathing circuit can be used in any type of pressurized breathing therapy including, for example, continuous positive air(way) pressure (CPAP) therapy and bilevel positive air pressure therapy where the inspiratory and expiratory pressures differ.

### FIELD

The present disclosure relates to a positive pressure breathing circuit and a method for ventilating a patient. The breathing circuit can be used in any type of pressurized breathing therapy including, for example, continuous positive air(way) pressure (CPAP) therapy and bi-level positive air pressure therapy where the inspiratory and expiratory pressures differ.

### BACKGROUND

Breathing circuits can help a patient to breathe by delivering gas to open up their airways and/or supplying specific breathing gases for a particular medicinal purpose. The breathing gases may be supplied at a flow rate that is higher than an average inspiratory flow rate to ensure there is no shortage of breathing gases. In the case of CPAP therapy, the flow supplied to the patient is usually higher than the peak inspiratory flow, rather than the average inspiratory flow.

Some traditional breathing circuits for ventilating a patient use a mixed breathing gas including a blend of air and oxygen gas that is supplied to a patient via an inspiratory tube. The required oxygen saturation levels in the patient's blood can be achieved by adjusting the ratio of the oxygen in the oxygen/air blend. However, a problem with this breathing circuit is the positive pressure experienced by the patient is the result of a continuous supply of the mixed breathing gas during both inhalation and exhalation, which results in a significant wastage of the oxygen gas. An improved breathing circuit allows increased amounts of oxygen gas to be delivered to the patient during inhalation to minimize its wastage during exhalation. However, the improved breathing circuits often required multiple pressure regulation devices to be carefully operated to allow exhaled gases to be vented from the circuit and to allow excess air supplied to also be vented simultaneously.

There is therefore a need for an alternative breathing circuit and a method.

### SUMMARY

An embodiment relates to a positive pressure breathing circuit for ventilating a patient, the breathing circuit comprising:
an inspiratory tube that is connectable to i) a patient interface for supplying a breathing gas, ii) a source of a pressurized first gas, and iii) a source of a pressurized second gas, wherein the second gas enters the inspiratory tube downstream to where the first gas enters the inspiratory tube; and
an expiratory tube configured to receive exhaled gas and vent gases including the exhaled gas from the patient interface;
wherein the inspiratory tube is connectable to the expiratory tube so that any excess of the first gas supplied to the inspiratory tube can be conveyed to the expiratory tube, and
the expiratory tube is configured so that the excess supply of the first gas conveyed to the expiratory tube and the exhaled gas received by the expiratory tube can be vented from the breathing circuit.

A distal portion of the inspiratory tube may be connectable to the source of the pressurized first gas.

A proximal portion of the inspiratory tube may be connectable to the source of the pressurized second gas.

The proximal portion of the inspiratory tube may be connectable to the patient interface.

The inspiratory tube may include a first non-return valve.

The first non-return valve may be arranged between the patient interface and the second gas entering the inspiratory tube.

The first non-return valve may be configured to inhibit the exhaled gas from entering the inspiratory tube. Throughout this specification, the first non-return valve inhibits the exhaled gas from passing upstream of the first non-return valve, but this does not necessarily mean that the first non-return valve completely blocks the flow.

The expiratory tube may include a second non-return valve.

The second non-return valve may be configured to inhibit the first gas from entering the patient interface from the expiratory tube. That is to say, to inhibit the excess supply of the first gas conveyed from the inspiratory tube from entering the patient interface from the expiratory tube. Throughout this specification, the second non-return valve inhibits the flow of the excess supply of the first gas passing upstream of the second non-return valve, which does not necessarily mean that the second non-return valve completely blocks the flow.

The expiratory tube may be configured so that the exhaled gas received by the expiratory tube downstream of the second non-return valve is vented from the breathing circuit.

The expiratory tube and the inspiratory tube may be connectable downstream of the second non-return valve.

The distal portion of the inspiratory tube may be connectable to the distal portion of the expiratory tube for conveying the excess supply of the first gas.

The proximal portions of the inspiratory and the expiratory tubes are connectable directly or indirectly with the patient interface to form a loop configuration.

The expiratory tube may be configured so that all of the excess supply of the first gas conveyed to the expiratory tube and all the exhaled gas in the expiratory tube are vented from the breathing circuit.

The expiratory tube may be configured so that all of the excess supply of the first gas conveyed to the expiratory tube downstream of the second non-return valve and all the exhaled gas in the expiratory tube downstream of the second non-return valve are vented from the breathing circuit.

The second non-return valve also inhibits the exhaled gas from being rebreathed.

The expiratory tube may have a substantially constant volume. That is to say in one example, the expiratory tube may not have a volume changing structure such as a bellows, collapsible chamber, or flexible walled passage or alike. The volume of the expiratory tube may fluctuate by a small amount due to pressure changes, but the macro structure of the expiratory tube is not configured to change with changes in pressure.

The expiratory tube may have a substantially constant volume upstream of the second non-return valve.

The expiratory tube may have a substantially constant volume downstream of the second non-return valve.

The inspiratory tube may have a substantially constant volume. The volume of the inspiratory tube may fluctuate by a small amount due to pressure changes, but the macro structure of the inspiratory tube is not configured to change with changes in pressure.

The inspiratory tube is configured so that a volume of the second gas can enter and be loaded in the inspiratory tube whilst the first gas can be supplied to the inspiratory tube, and the first gas supplied in excess can be conveyed to the expiratory tube and vented from the expiratory tube. This can occur during patient exhalation.

The inspiratory tube is configured so that the second gas can enter the inspiratory tube whilst the first gas can be supplied to the inspiratory tube, and the first gas supplied in excess can be conveyed to the expiratory tube and vented from the expiratory tube. This can occur during patient inhalation and exhalation.

In one example, the breathing circuit comprises a bypass tube interconnecting the inspiratory tube and the expiratory tube that conveys the first gas from the inspiratory tube to the expiratory tube.

In another example, the inspiratory tube and the expiratory tube are directly interconnected. In this instance, the breathing circuit may include a first gas connector that interconnects the inspiratory tube and the expiratory tube. The first gas connector may, for example, include a multi limb joiner such as a Y-shaped joiner, a T-shaped joiner and so forth, and a manifold having one or two inlets, and one or more outlets.

The inspiratory tube may have a first gas inlet for the first gas, the first gas inlet may be configured so that the first gas enters laterally to the inspiratory tube and parallel or coaxial to the bypass tube.

The first gas inlet may include a first tube connector having multiple limbs, including a first limb that is connectable to a first gas source, a second limb that is connectable to the inspiratory tube, and a third limb that is connectable directly or indirectly to the expiratory tube. Indirect connection may be provided by the bypass tube interconnecting the inspiratory tube and the expiratory tube.

The second limb of the first tube connector may be arranged laterally to the first limb, and the third limb may be arranged linearly with the first limb. The first tube connector provides flow resistance to the first gas entering the inspiratory tube.

The inspiratory tube may have a second gas inlet for the second gas, the second gas inlet may be configured so that the second gas enters the inspiratory tube lateral to a longitudinal axis of the inspiratory tube. The second gas inlet may be arranged upstream of the first non-return valve.

The second gas inlet may include a second tube connector having multiple limbs, including a first limb that is connectable to a second gas source, a second limb that is connectable to the inspiratory tube extending toward the first non-return valve, and a third limb that is connectable to the inspiratory tube that extends upstream of the second gas inlet.

The inspiratory tube and the expiratory tube may have a continuous open line so the first gas can be conveyed from the inspiratory tube to the expiratory tube in one direction.

In the situation where the bypass tube interconnects the inspiratory tube and the expiratory tube, the bypass tube may be connected to the expiratory tube by a third tube connector having multiple limbs, including a first limb that is connectable to the bypass tube, a second limb that is connectable to a distal portion of the expiratory tube downstream of the second non-return valve, and the third limb is connected to the remainder of the distal portion of the expiratory tube extending away from the second non-return valve. The third limb is arranged parallel to, or co-axially with, the first limb, and the second limb is arranged laterally to the first limb. That is to say, the first and third tube connectors are configured to allow the first gas to be conveyed therethrough to provide less flow resistance to the first gas flowing from the first gas source to the expiratory tube compared to the flow resistance to the first gas entering and flowing along the inspiratory tube.

The first gas received by the expiratory tube is vented from the breathing circuit without being accumulated or stored, and the expiratory tube is configured so that the exhaled gas passes through the second non-return valve and is vented from the breathing tube without being accumulated or stored.

The expiratory tube may be configured so that the first gas and the exhaled gas downstream of the second non-return valve are vented from the breathing circuit without re-entering the inspiratory tube.

The breathing circuit may be configured so that there is greater flow resistance for the first gas from the inspiratory tube to the expiratory tube via the patient interface than the flow resistance for the excess of the first gas from the inspiratory tube to the expiratory tube.

The breathing circuit also reduces inefficient use of the second gas by preventing it from continuously passing through the patient interface. This is achieved primarily by the first non-return valve being closed during exhalation. In addition, the breathing circuit has flow resistance that inhibits the flow of the second gas from the inspiratory tube to the expiratory tube by the pressure drop across the first and second non-return valves, and the pressure drop over the lengths of the inspiratory tube and the expiratory tube.

The inspiratory tube may be configured so that the first gas and the second gas entering the inspiratory tube inhibits the exhaled gas from entering the inspiratory tube. For example, the inspiratory tube may have an open passageway.

The breathing circuit may comprise a pressure regulation device configured to regulate pressure in the expiratory tube.

The pressure regulation device may include a pressure relief valve configured to vent the first gas and the exhaled gas from the expiratory tube. The pressure relief valve of the expiratory tube may be a passive valve. For example, the pressure relief valve may be a positive end expiratory pressure valve having a fixed operating pressure or an operating pressure that can be manually adjusted. That is to say, the valve does not require active control measures or an actuator to continually monitor and adjust the operating pressure of the valve.

The pressure regulation device may include a positive end expiratory pressure valve (PEEP valve) on the distal portion of the expiratory tube.

The positive end expiratory pressure valve of the expiratory tube may have a pressure setting ranging from about 2.5 to 20.0 cmH₂O, or ranging from about 8.0 to 12.0 cmH₂O, or about 10.0 cmH₂O.

The first non-return valve may be arranged downstream of where the second gas enters the inspiratory tube. That is, the first non-return valve may be between the patient interface and where the second gas enters the inspiratory tube.

The first non-return valve may be located adjacent to the patient interface.

The first non-return valve may be located proximal to where the second gas enters the inspiratory tube.

The breathing circuit may also include a gas flow generator that supplies the pressurized first gas; and a sensor that senses when the patient breathes, the sensor having an output signal that is used to operate the gas flow generator

The sensor may include a gas meter in the expiratory tube. That is to say, the gas meter measures a property of the gas in the expiratory tube and the output of the gas meter is used to operate the flow generator. The property of the gas meter may be any suitable property including gas flow rate, gas pressure, gas temperature, gas humidity or gas concentration, such as oxygen or carbon dioxide concentration.

The sensor may include a flow sensor located upstream of the second non-return valve and a pressure sensor located downstream of the second non-return valve. That is to say, the flow sensor measures the flow of the exhaled gas in the expiratory tube and the pressure sensor measures the pressure of the exhaled gas and the first gas being vented from the expiratory tube.

The breathing circuit may include a controller that receives the outputs of the flow and pressure sensors, and the controller has a processor that calculates a control output that is used to operate the flow generator and adjust the gas flow generator to target a desired pressure.

The inspiratory tube may have a length ranging from about 0.5 m to 2.5 m, or about a length ranging from 0.75 to 2.0 m, or a length ranging from about 1.5 to 1.8 m. The inspiratory tube may include a gas passageway of constant diameter, in which the diameter may range from about 18 to 25 mm, or the diameter is about 22 mm.

The inspiratory tube may have an internal volume ranging from about 100 ml to 760 mL, for storing the second gas and some of the first gas. For example, the internal volume of the inspiratory tube may be about 315, 350 or 500 mL.

In further examples, the internal volume may range from about 315 mL to 760 mL for adult patients, or range from about 400 to 600 mL. For pediatric patients, the internal volume may range from about 100 mL to 450 mL, or range from about 200 to 400 mL. For neonatal patients, the internal volume may range from about 50 to 200 mL, or range from about 100 to 150 mL.

The inspiratory tube ideally has an internal volume that allows the pressurized oxygen gas that is stored in the inspiratory tube to be inhaled by the patient in a single inhalation so that venting of the pressurized oxygen gas from the inspiratory tube during exhalation can be avoided, thereby minimizing wastage of the pressurized oxygen gas.

The volume of the pressurized oxygen gas that may enter the inspiratory tube during patient exhalation may range from about 50 to 90 percent by vol % of a tidal volume of a patient, or range from about 60 to 70 percent by vol % of a tidal volume of a patient.

The volume of the pressurized oxygen gas that may enter the inspiratory tube during patient exhalation can equal an estimation of an alveoli volume of the patient.

The first gas may be pressurized air. The first gas may be pressurized air enriched with oxygen.

In one example, the second gas may be pressurized oxygen gas.

In another example, the second gas may be a pressurized gas including one or any combination of: oxygen gas, heliox, or an anaesthetic gas. The anaesthetic gas could be nitrous oxide or a 50:50 mixture of nitrous oxide and oxygen gas.

Pressurized oxygen gas may be supplied from a liquified oxygen source, a bottled oxygen source or from an oxygen concentrator source.

The breathing circuit may include a patient interface. The patient interface may be a sealed patient interface. The first non-return valve may be arranged on the patient interface.

The patient interface may have an inlet connection that connects to the inspiratory tube, and an outlet connection that connects to the expiratory tube.

The patient interface may have a coupling to which a Y-piece is or can be connected, in which one leg of the Y-piece is an inlet connection that connects to the inspiratory tube, and another leg is an outlet connection that connects to the expiratory tube.

The inspiratory tube may be directly connected to the patient interface either with or without a Y-piece. That is to say, there are no intervening operations such as humidifiers, heat and moisture exchangers, or other items that have the potential to increase dead space in the breathing circuit between the inspiratory tube and the patient interface.

An embodiment relates to a positive pressure breathing circuit for ventilating a patient, the breathing circuit comprising:
a loop configuration that is connectable to a patent interface, the loop configuration includes an inspiratory tube and an expiratory tube, the inspiratory tube being connectable to: i) a patient interface, ii) a first gas source, and iii) a second gas source, wherein the second gas enters the inspiratory tube downstream to where the first gas enters the inspiratory tube;
wherein the inspiratory tube is connected to the expiratory tube so that the first gas supplied in excess to the breathing circuit is received by the expiratory tube and vented with the exhaled gas from the breathing circuit.

A distal portion of the inspiratory tube may be connectable to the source of the pressurized first gas.

A proximal portion of the inspiratory tube may be connectable to the source of the pressurized second gas.

The proximal portion of the inspiratory tube may be connectable to the patient interface.

The breathing circuit may include a first non-return valve arranged in the inspiratory tube downstream of where the second gas source connects to the inspiratory tube.

The breathing circuit may include a second non-return valve arranged in the expiratory tube.

The loop configuration may include the first tube connector having three limbs. In one example, the first limb is connectable to the first gas source, a second limb is connectable to a distal portion of the inspiratory tube, and a third limb is connectable to the expiratory tube.

In another example, the loop configuration may have a bypass tube interconnecting the inspiratory limb and the expiratory limb. The bypass tube may be connected to the inspiratory tube using any suitable three limb connector.

In situations where the loop configuration has the bypass tube, the second connection limb may connect the bypass tube to the expiratory tube.

An embodiment relates to a device that can be arranged between a gas non-return valve and a gas tube, the device includes a body having a bay portion that connects to the non-return valve, and a flow director extending from the bay portion that receives gas from the gas tube, wherein the flow director has a flow constriction that is configured to increase speed of the gas passing therethrough and faces toward the non-return valve so that the gas that exits the flow director assists in biasing the non-return valve into an operating position.

The bay portion may be fixedly connected to an outlet of the non-return valve.

The bay portion may be removably connected to an outlet of the non-return valve.

The non-return valve may be the second non-return valve described herein.

The bay portion may be fixedly connected to the gas tube. In one example, the gas tube may be the bypass tube described herein.

The bay portion may be removably connected to the gas tube.

The operating position of the non-return valve may be a closed position.

The flow constriction may have a nozzle that faces toward the non-return valve.

The flow constriction may have a passageway that narrows in a direction of flow of the first gas.

The flow constriction may have converging walls in the direction of flow of the first gas.

The flow constriction has a discharge portion having an outlet for discharging the gas passing through the flow director, in which the discharge portion has a constant diameter.

The flow constriction may include a converging portion that narrows in a direction of flow of the first gas.

The flow constriction may have an outlet orifice that faces toward the second non-return valve. The outlet orifice may have cross-sectional area ranging from about 10 to 80% less than a cross-section area of the inspiratory tube, and suitably ranging from about 20 to 70% less, and suitably ranging from about 30 to 60% less, and suitably ranging from about 40 to 50% less than a cross-section area of the inspiratory tube.

The non-return valve and the flow director may be oppositely disposed on the bay portion.

The body may have an outer wall that defines the bay portion.

The body may have an outer wall having opposite ends that connect to the non-return valve and the gas tube, the outer wall also defining the bay portion as a cavity between the opposite ends.

The device may have a tubular formation extending from the outer wall in which the flow director is located.

The body of the device may have a discharge outlet for discharging the gas passing through the flow director and the non-return valve. That is to say, the non-return valve has an outlet that opens into the bay portion. The discharge outlet may extend from the outer wall.

The discharge outlet may be integrally formed with the expiratory tube described herein.

The device may be included in the breathing circuit described herein. For example, the first gas in the bypass tube, may pass through the flow director to help bias the non-return valve, suitably the second non-return valve in a closed position during patient inhalation.

An embodiment relates to a positive pressure breathing circuit for ventilating a patient, the breathing circuit including:
an inspiratory tube including a distal portion that is connectable to: i) a source of a pressurized first gas, ii) a source of a pressurized second gas, and iii) a patient interface for supplying a breathing gas, wherein the second gas enters the inspiratory tube downstream to where the first gas enters the inspiratory tube;
an expiratory tube configured to receive exhaled gas and vent gases including the exhaled gases from the patient interface;
wherein the inspiratory tube is connectable to the expiratory tube so that any excess of the first gas supplied to the inspiratory tube can be conveyed from the inspiratory tube to the expiratory tube; and
wherein the expiratory tube includes a flow director for the first gas, the flow director having a flow outlet that faces toward the second non-return valve to help bias the second non-return valve into a closed position.

A distal portion of the inspiratory tube may be connectable to the source of the pressurized first gas.

A proximal portion of the inspiratory tube may be connectable to the source of the pressurized second gas.

The proximal portion of the inspiratory tube may be connectable to the patient interface.

The expiratory tube may include a second non-return valve.

The inspiratory tube may include a first non-return valve that is arranged between the patient interface and the second gas entering the inspiratory tube.

Flow of the first gas through the flow director may fluctuate, for example, cycle from a higher flow when the patient exhales, meaning the patient exhaling will need to overcome any bias of the second non-return valve in the closed position.

The flow director may be a constriction in the expiratory tube that opens toward the second non-return valve.

The flow director may be a nozzle.

The flow director may have a passageway that narrows in a direction of flow of the first gas, in which the passageway has an opening that faces toward the second non-return valve. The purpose of the flow director is to direct the first gas exiting the flow director at an increased speed to impact on the second non-return valve, thereby providing additional biasing to close the second non-return valve. In other words, the excess first gas can provide a velocity head for closing the second non-return valve.

An embodiment relates to a positive pressure breathing circuit for ventilating a patient, the breathing circuit comprising:
an inspiratory tube with a gas passageway, the inspiratory tube being connectable to: i) a patient interface for supplying a breathing gas, ii) a pressurized first gas for supplying the first gas, and iii) a source of a pressurized second gas, wherein the second gas enters the inspiratory tube downstream to where the first gas enters the inspiratory tube;
an expiratory tube configured to receive exhaled gas and vent gases including the exhaled gases from the patient interface;
wherein the inspiratory tube is connectable to the expiratory tube so that any of the first gas supplied to the inspiratory tube supplied in excess flows from the inspiratory tube to the expiratory tube; and
a sensor that senses when the patient breathes, the sensor having an output signal that is used to operate a gas flow generator for the first gas.

A distal portion of the inspiratory tube may be connectable to the source of the pressurized first gas.

A proximal portion of the inspiratory tube may be connectable to the source of the pressurized second gas.

The proximal portion of the inspiratory tube may be connectable to the patient interface.

The breathing circuit may include a gas flow generator that supplies the first gas.

The inspiratory tube may include a first non-return valve that is arranged downstream of the second gas entering the inspiratory tube.

The first non-return valve may be configured to inhibit the exhaled gas from entering the inspiratory tube.

The expiratory tube may include a second non-return valve.

The second non-return valve may inhibit the first gas from entering the patient interface from the expiratory tube.

The gas flow generator may be a variable flow generator that is operable at a higher pressure and a lower pressure. Typically the gas flow generator operates at the higher pressure when the sensor detects inhalation, and at the lower pressure when the sensor detects exhalation. The flow generator may cycle between the higher pressure and the lower pressure during continuous patient breathing. That is to say, the positive pressure breathing circuit may be a bi-level breathing circuit for bi-level positive air pressure therapy, also known as BiPAP. However, it will be appreciated by those skilled in the art that the pressure levels are nominal and that the breathing circuit will be operated over a pressure range.

The sensor may include a flow sensor located upstream of the second non-return valve and a pressure sensor located downstream of the second non-return valve. That is to say, the flow sensor measures the flow of the exhaled gas in the expiratory tube and the pressure sensor measures the pressure of the exhaled gas and the first gas being vented from the expiratory tube.

The breathing circuit may include a controller that receives the outputs from the flow sensor and the pressure sensors, and the controller has a processor that calculates a control output that is used to operate the flow generator and adjust the flow generator to target a desired pressure.

Another embodiment relates to a method of ventilating a patient, the method including steps of:
a) providing a positive pressure breathing circuit including:
   an inspiratory tube that is connectable to: i) a patient interface, ii) a source of a pressurized first gas, and iii) a source of a pressurized second gas, wherein the second gas enters the inspiratory tube downstream to where the first gas enters the inspiratory tube;
   an expiratory tube configured to receive exhaled gas and vent gases including the exhaled gases from the patient interface;
   wherein the inspiratory tube is connectable to the expiratory tube; and
b) supplying a pressurized second gas into the proximal portion of the inspiratory tube; and
c) supplying the pressurized first gas into the distal portion of the inspiratory tube, and during patient exhalation a volume of the pressurized second gas that enters and is stored in the inspiratory tube displaces the first gas from the inspiratory tube and is conveyed to the expiratory tube.

The step of supplying the first gas to the inspiratory tube includes supplying in an excess amount and conveying excess supply of the first gas from the inspiratory tube to the expiratory tube, and venting the excess supply of the first gas and the exhaled gas from the expiratory tube.

The inspiratory tube may include a first non-return valve.

The first non-return valve may inhibit the exhaled gas from entering the inspiratory tube.

The expiratory tube may include a second non-return valve.

The second non-return valve may inhibit the first gas from entering the patient interface from the expiratory tube.

Supplying the pressurized second gas into the proximal portion of the inspiratory tube may include the second gas entering the inspiratory tube upstream from the first non-return valve.

The method may further include the step of venting the first gas and the exhaled gas received by the expiratory tube from the breathing circuit.

The first gas and the exhaled gas may be vented from the breathing circuit downstream of the second non-return valve.

The step of supplying the pressurized first gas may be carried out continuously to the distal portion of the inspiratory tube.

The step of supplying the pressurized first gas may be carried out at a rate that is greater than or equal to peak inspiratory flow rate of a patient.

The step of providing the breathing circuit includes the breathing circuit having a pressure regulating device, and the method includes regulating the pressure in the breathing circuit.

The pressure regulating device may be a positive end expiratory valve (PEEP valve) in the expiratory tube downstream of the second non-return valve, and the method may include operating the PEEP valve to vent the exhaled gas and the first gas from the breathing circuit at a desired pressure.

The method may include selecting a pressure setting of the PEEP valve of the expiratory tube within a range from about 2.5 to 20.0 cmH₂O, or a range from about 8.0 to 12.0 cmH₂O, or about 10.0 cmH₂O.

The step of supplying the second gas may include controlling the flow rate of the second gas to the inspiratory tube at a rate depending on the requirements of the patient. For instance, when the second gas is oxygen gas the flow rate will depend on the oxygen saturation concentration of the patient's blood. In one example, the controlling the flow rate of the second gas may include supplying the second gas at a constant rate.

In another example, controlling the flow rate of the first gas may include cycling output pressure of the flow generator between high pressure during patient inhalation and low pressure during patient exhalation. For example, controlling the flow rate of the first gas may include cycling output pressure of the flow generator between high pressure flow and low pressure flow based on inhalation of the patient and exhalation, respectively.

The breathing circuit provided may include a gas flow sensor in the expiratory tube upstream of the second non-return valve and a pressure sensor located downstream of the second non-return valve, and the method may include detecting when the patient is exhaling based on an output of the flow sensor and detecting the pressure of first gas being supplied to the breathing circuit based on an output of the pressure sensor.

The breathing circuit may include a controller that receives the outputs of the gas flow and pressure sensors, and the controller has a processor that calculates a control output that is used to operate the flow generator and adjust the flow generator to target a desired pressure.

The controller may detect a trigger gas flow rate shortly after the start of the patient exhaling and toward the end of the patient exhaling. The trigger gas flow rate may be in the range from about 3 to 15% of the maximum exhaling flow rate, and suitably about 5 to 10% of the maximum exhaling flow rate.

The flow rate of the second gas may be controlled independently of any one or any combination of:
I. the tidal flow of the patient;
II. changes in tidal flow of the patient;
III. a flow rate at which the pressurized air is supplied into the inspiratory tube; or
IV. pressure changes within the breathing circuit.

In other words, the flow rate of the second gas supplied to the inspiratory tube can be determined so that the volume of the second gas stored in the inspiratory tube during exhalation and supplied to the inspiratory tube during inhalation will occupy the alveoli volume of the patient, meaning that enriched second gas need not be vented from the breathing circuit or drawn into dead space of the patient or of the breathing circuit. As a result, there will be less wastage of the second gas by reducing venting of the second gas from the breathing circuit without being inhaled, and by having a higher portion of the second gas in the alveoli volume of a patient's lungs than in the dead space.

In the situation where the second gas includes enriched oxygen gas, controlling the flow rate of the oxygen gas supplied to the inspiratory tube may be based on a level of oxygen saturation in the patient's blood.

During patient exhalation, the second gas entering the inspiratory tube can flow backwards along the inspiratory tube which acts as a constant pressure storage volume by displacing air out of the inspiratory tube via the further positive end expiratory pressure valve of the inspiratory tube.

During patient inhalation, the breathing gas from the inspiratory tube will initially be the second gas that had been stored in the inspiratory tube and then the first gas. That is to say, during patient inhalation, the breathing gas the patient initially receives includes the second gas that was stored in the inspiratory tube before the first gas.

When the first gas is air, supplying the air may be carried out in the range from about 2 to 120 L/min.

For example, in the case of an adult patient, the air may be supplied to the inspiratory tube at a range from about 40 to 120 L/min, or range from about 50 to 70 L/min. In the case of pediatric patients, the air may be supplied to the inspiratory tube at a range from about 3 to 50 L/min, or a range from about 4 to 40 L/min. In the case of neonatal patients, air may be supplied to the inspiratory tube at a range from about 2 to 10 L/min, or at a range from about 3 to 6 L/min.

The inspiratory tube may have a length ranging from about 0.5 m to 2.5 m, or a length ranging from about 0.75 to 2.0 m, or a length ranging from about 1.5 to 1.8 m. The inspiratory tube may include a gas passage of constant diameter, in which the diameter may range from about 18 to 25 mm, or a diameter about 22 mm.

The inspiratory tube may have an internal volume ranging from about 100 mL to 760 mL, for storing the second gas and some of the first gas. For example, the internal volume of the inspiratory tube may be about 315, 350 or 500 mL.

In further examples, the internal volume may range from about 315 mL to 760 mL for adult patients, or range from about 400 to 600 mL. For pediatric patients, the internal volume may range from about 100 mL to 450 mL, or range from about 200 to 400 mL. For neonatal patients, the internal volume may range from about 50 to 200 mL, or range from about 100 to 150 mL.

The breathing circuit provided by the method described herein may include any one or a combination of the features described herein. Similarly, the method described herein may include any one or a combination of the features of the breathing circuit.

The embodiments described in the paragraphs [0005], [0066], [0075], [0095], [0105] and [0118] may include any one or a combination of the features described herein.

Throughout this specification the term “excess supply of the first gas”, or variations thereof, refers to an amount of the first gas supplied by the flow generator that is not delivered to the patient interface.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 is a schematic illustration of a positive pressure breathing circuit for ventilating a patient, in which a pressurized first gas is supplemented using a pressurized second gas and the first gas is supplied to both an inspiratory tube and an expiratory tube.

FIG. 2 is a graph illustrating possible flow rates in the breathing circuit that can be supplied to a patient interface and discharged from the patient interface during inhalation and exhalation, and the dotted line represents the constant supply of the second gas to the breathing circuit.

FIG. 3 is a schematic illustration of a positive pressure breathing circuit for ventilating a patient, in which a pressurized first gas is supplemented using a pressurized second gas and the first gas is supplied to an inspiratory tube and an expiratory tube.

FIG. 4 is an enlarged view of a portion of the breathing circuit circled in FIG. 3, the enlarged view illustrating a portion of expiratory tube in cross-section.

FIG. 5 is a schematic illustration of a positive pressure breathing circuit for ventilating a patient, in which a pressurized first gas is supplemented using a pressurized second gas as shown in FIG. 1 and the breathing circuit having at least one sensor and flow control for controlling the flow of the first gas to the breathing circuit based on an output of the sensor.

FIG. 6 is a schematic illustration of a positive pressure breathing circuit for ventilating a patient as shown in FIG. 5, in which the breathing circuit has two sensors that are used to operate a flow generator.

FIG. 7A is a graph illustrating an exhalation pattern of a patient shown by the solid line, and a dotted line intersecting the solid line representing a trigger point used by the controller in FIGS. 5 and 6.

FIG. 7B is a graph illustrating the upper and lower set points of a controller that are assigned to the patient to operate a flow generator.

FIG. 8 is a block diagram of method steps for ventilating a patient using the breathing circuit shown in FIGS. 1, 3, 5 and 6.

FIG. 9 is a block diagram of method steps for controlling the supply of the first gas to the breathing circuit.

### DETAILED DESCRIPTION

An embodiment will now be described in the following text which includes reference numerals that correspond to features illustrated in the accompanying Figures. To maintain clarity of the Figures, however, not all reference numerals are included in each Figure. Although certain examples are described herein, those of skill in the art will appreciate that the disclosure extends beyond the specifically disclosed examples and/or uses and obvious modifications and equivalents thereof. Thus, it is intended that the scope of the disclosure herein disclosed should not be limited by any particular examples described herein.

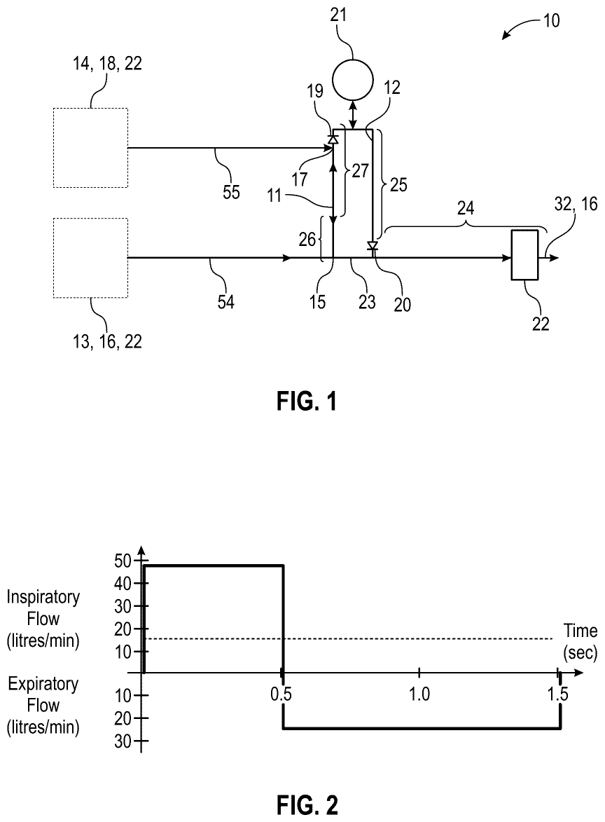

FIGS. 1, 3, 5 and 6 illustrate a positive pressure breathing circuit 10 for ventilating a patient. The breathing circuit 10 can be used for different breathing therapies, including Continuous Positive Airway Pressure (CPAP) therapy and bi-level positive air(way) pressure therapy where the inspiratory and expiratory pressures differ.

The breathing circuit 10 includes an inspiratory tube 11 having a gas passageway that conveys gas toward a sealed patient interface 21 and an expiratory tube 12 also having a gas passageway that conveys gas away from the patient interface 21. As can be seen in FIGS. 1, 3, 5 and 6, distal portions 26 and 24 of the inspiratory tube 11 and the expiratory tube 12 are interconnected, and proximal portions 27 and 25 of the inspiratory and expiratory tubes 11, 12 are interconnected to form a loop configuration. A first gas 16 is supplied to both the distal portion 26 of the inspiratory tube 11 and to a distal portion 24 of the expiratory tube 12 from a first gas source 13. Specifically, the first gas 16 is supplied to the distal portion 26 of the inspiratory tube 11, and any excess of the first gas 16 supplied to inspiratory tube 11 is then conveyed to the expiratory tube 12. The interconnection between the inspiratory and expiratory tubes 11, 12 may be a direct connection by way of a suitable coupling device or a joiner, such as two or more connectors having three connection limbs, a manifold arrangement which are not shown in the Figures. The interconnection between the inspiratory and expiratory tubes 11, 12 may be an indirect connection, an example of which as shown in the Figures as a bypass tube 23 that conveys the first gas 16 supplied in excess from the inspiratory tube 11 to the expiratory tube 12. The expiratory tube 12 vents exhaled gas 32 from the patient interface 21 and excess supply of the first gas is conveyed by the bypass tube 23 to the expiratory tube 12 so that the excess supply of the first can be vented.

The inspiratory tube 11 has a proximal portion 27 close to the patient that is connectable to the sealed patient interface 21 for supplying a breathing gas, and a distal portion 26 more remote from the patient that has a first gas inlet 15 that is connectable to the first gas source 13. The proximal portion 27 of the inspiratory tube 11 also includes a second gas inlet 17 that is connectable to a second gas source 14 of a pressurized second gas 18 and includes a first non-return valve 19 that is located upstream of the patient interface 21 and downstream of the second gas inlet 17 where the second gas 18 enters the inspiratory tube 11. The first non-return valve 19 inhibits exhaled gas 32 from entering the inspiratory tube 11 and is located in the proximal portion 27 of the inspiratory tube 11 and is located proximal to the second gas inlet 17. In addition, the first non-return valve 19 inhibits the second gas passing to the patient interface 21 during patient exhalation. In another example, not illustrated, the first non-return valve 19 may be located on the patient interface 21.

FIGS. 1, 3, 5 and 6 depict the expiratory tube 12 and the inspiratory tube 11 being connected to the patient interface 21 by a tube connector having three limbs, such as a Y-piece, in which one of the limbs connects to the expiratory tube 12, another limb connects to the inspiratory tube 11, and a third limb of the tube connector couples to an inlet/outlet port on the patient interface 21 to conduct breathing gas as it is inhaled and exhaled. The tube connector may be integrally formed with the patient interface 21. Alternatively, the tube connector may be separate from the patient interface 21, and either manually connected to a connection port of the interface 21 or a length of the tubing can interconnect the tube connector and a connection portion of the patient interface 21. In another example, not illustrated in the Figures, the inspiratory tube 11 and the expiratory tube 12 may be directly connected to an inlet connection of the patient interface 21 and an outlet connection of the patient interface 21 respectively.

The expiratory tube 12 includes a second non-return valve 20 that inhibits exhaled gas 32 from re-entering the patient interface 21 after having been exhaled and inhibits the first gas 16 from entering the patient interface 21 from the expiratory tube 12. As can be seen in FIGS. 1, 3, 5 and 6, the second non-return valve 20 is located in the distal portion 24 of the expiratory tube 12.

The first and second non-return valves 19, 20 may be any suitable valves, include a one-way flap valve, a biased valve that is biased into a closed position, or a diaphragm valve. The first non-return valve 19 closes when the gas pressure downstream of the first non-return valve 19, for instance in the patient interface 21, is greater than the pressure in the inspiratory tube 11. The first non-return valve 19 opens, suitably automatically, when the patient spontaneously inhales. More particularly during patient inhalation, the first non-return valve 19 is in an opened position and the second non-return valve 20 is in a closed position. Similarly, the second non-return valve 20 opens when the patient spontaneously exhales, so that during patient exhalation, the first non-return valve 19 is in a closed position and the second non-return valve 20 is in the opened position. The first and second non-return valves 19 and 20 provide flow resistance when in the closed position and to a degree when in the opened position. In practice this means that first non-return valve 19 has flow resistance even when in the opened position, and similarly, the second non-return valve 20 has flow resistance even when in the opened position. The flow resistance provided by the first and second non-return valves 19 and 20 reduces the risk of the first gas 16 supplied in excess passing through the inspiratory tube 11 and out through the expiratory tube 12 via the patient interface 21, which if this occurred would reduce the efficiency in use of the second gas 18.

In addition, when the patient exhales, the pressure at which the patient spontaneously exhales, which does not need to be higher than the pressure of the second gas source 14 can cause the first non-return valve 19 to change to the closed position. This further reduces the risk of the first gas 16 supplied in excess passing through the inspiratory tube 11 and out through the expiratory tube 12 which if occurred, would reduce the efficiency of the second gas used by the breathing circuit.

In FIGS. 1, 3, 5 and 6, the first gas inlet 15 is a first T-shaped tube connector having three limbs. Specifically, a first limb is connected to the first gas source via a first supply line, a second limb is connected to a distal portion 26 of the inspiratory tube 11 in which the second limb is arranged laterally to the first gas supply line 54, and a third limb is connected to the bypass tube 23. The third limb is arranged parallel to, or co-axially with, the first limb. One of the advantages in the configuration of the first tube connector is that the first gas must turn about a corner, such as a 90 degree corner, when flowing into the inspiratory tube 11, whereas the first gas 16 is not required to change direction in the tube connector in flowing toward the expiratory tube 12. As such the first tube connector provides some flow resistance to reduce the likelihood of the first gas passing from the inspiratory tube 11 to the expiratory tube 12 via the patient interface without being inhaled so that all excess flow of the first gas 16 passes to the expiratory tube 12. If the first gas 16 did pass from the inspiratory tube 11 to the expiratory tube 12 via the patient interface 21 this would reduce the efficiency of the second gas used by the breathing circuit

The second gas inlet 17 is a second T-shaped tube connector having three limbs. Specifically, a first limb is connected to a second gas source via a second supply line 55, a second limb is connected to a proximal portion 27 of the inspiratory tube 11 that includes the first non-return valve 19, the second limb being lateral to the first limb, and a third limb is connected to the inspiratory tube 11 that extends upstream of the second gas inlet 17. The third limb is arranged parallel to, or co-axially with, the second limb.

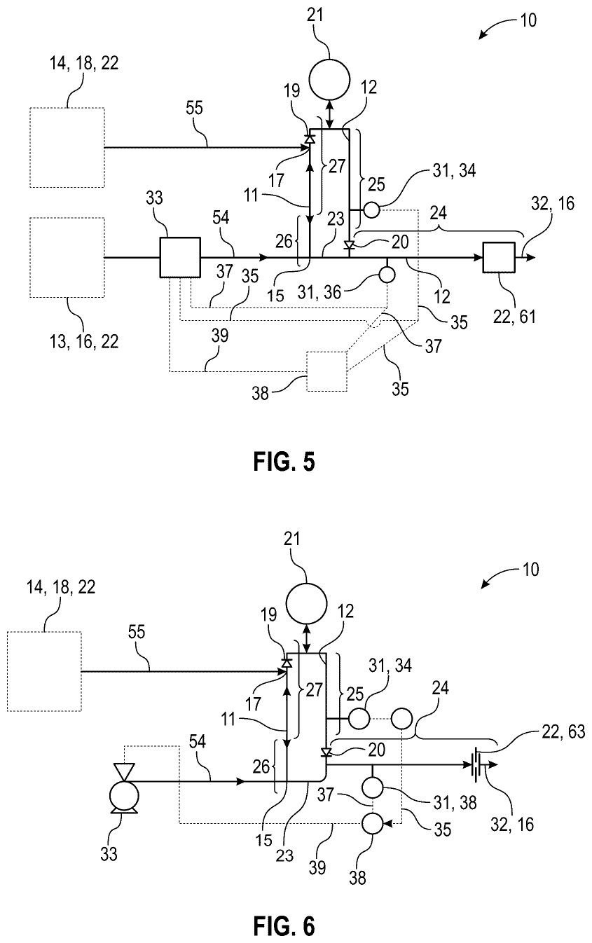

In FIGS. 1 and 5, the bypass tube 23 is connected to the expiratory tube 12 by a third T-shaped tube connector having three limbs. Specifically, a first limb is connected to the bypass tube 23, a second limb is connected to a distal portion 24 of the expiratory tube 12 immediately downstream of the second non-return valve 20, and the third limb is connected to the remainder of the distal portion 24 of the expiratory tube 12 extending away from the second non-return valve 20. The third limb is arranged parallel to, or co-axially with, the first limb, and the second limb is arranged laterally to the first limb. One of the advantages in the configuration of the third tube connector is that any gas flowing from the patient interface 21 down the expiratory tube 12 is required to turn about a corner, such as a 90 degree corner in order to be vented. Gas flowing from the bypass tube 23 to the expiratory tube 12 is not required to turn a corner. This configuration further reduces the likelihood of the first gas 16 passing from the inspiratory tube 11 to the expiratory tube 12 via the patient interface 21 without being inhaled, such that all excess flow of the first gas 16 passes to the expiratory tube 12 via the bypass tube 23.

In FIG. 6, the bypass tube 23 is connected to the expiratory tube 12 by a third T-shaped tube connector having three limbs. Specifically, a first limb is connected to the bypass tube 23, a second limb is connected to a distal portion 24 of the expiratory tube 12 immediately downstream of the second non-return valve 20, and the third limb is connected to the remainder of the distal portion 24 of the expiratory tube 12 extending away from the second non-return valve 20. The first limb is arranged parallel to, or co-axially with, the second limb, and the third limb is arranged laterally to the first limb (and the second limb). In the case of the third tube connector the exhaled gas 32 and the excess supply of the first gas 16 are required to turn about a corner, such as a 90 degree corner in order to be vented, and the first gas 16 in turning the corner can apply some pressure or head to the second non-return valve 20 to potentially assist in closing the second non-return valve 20 during patient inhalation. This further reduces the likelihood of the excess supply of the first gas 16 passing from the inspiratory tube 11 to the expiratory tube 12 via the patient interface 21 during inhalation, which could detrimentally displace the second gas 18 from the patient interface 21 if there was an unexpected leakage at the patient interface 21 or in the expiratory tube 12.

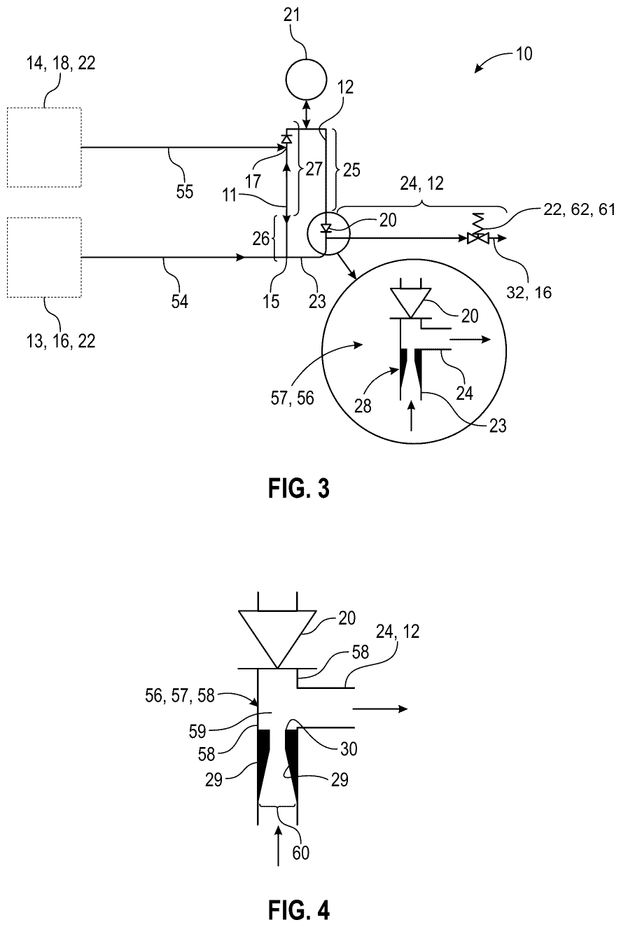

In the case of FIG. 3, the breathing circuit 10 includes a device 56 having a flow director 28, such as a nozzle that has a narrowing passageway 29 moving in a direction of the flow of the first gas 16. The device 56 may be removably attached to the breathing circuit 10, such as a three limbed tube connector, or the device 56 may be integrally connected to one or more adjacent parts of the breathing circuit 10, such the bypass tube 23, the second non-return valve 20, or the distal section of the expiratory tube 24 downstream of the device 56. As such, the breathing circuit 10 shown in FIG. 6 may optionally also include the device 56 shown in FIG. 4. For example, instead of, or in addition to, the third tube connector.

An enlarged cross-sectional view of the device 56 is shown in FIG. 4. The device 56 has a body 57 including an outer wall 58 that defines a bay portion 59 that connects to the second non-return valve 20 and connects to the flow director 28 that is oppositely disposed to the second non-return valve 20. In the example illustrated the flow director 28 has a flow constriction that reduces the cross-sectional of the flow passageway, thereby increasing the speed of the first gas. As can be seen, the flow director 28 has an entrance section 60 defined by inwardly tapering side walls 29 and an end section having a constant cross-section defining a trailing orifice 30. The purpose of the flow director 28 is to direct the first gas 16 exiting the flow director 28 at an increased speed to directly impact on the second non-return valve 20, thereby providing an additional pressing force to close the second non-return valve 20. In other words, the excess first gas 16 can provide a velocity head for closing the second non-return valve 20. One of the advantages provided by the flow director 28 is that between inhalations, such as during exhalation, or during pauses between exhalation and inhalation, excess supply/flow of the first gas 16 impacting on, or even facing toward the second non-return valve 20 helps close the second non-return valve 20. This increases the likelihood of the second non-return valve 20 fully closing during patient inhalation, and in turn reduces the likelihood of the first gas 16 and the second gas 18 passing through or bypassing through the patient interface 21 when the patient is not inhaling.

The breathing circuit 10 also includes a pressure regulation device 22 for regulating the pressure in the breathing circuit 10. The pressure regulation device 22 includes, in part, the inherent pressure of the first gas source 13 and/or a control valve (not illustrated) that may throttle pressure of the first gas 16 delivered to the inspiratory tube 11 by the first gas source 13 and, optionally, the flow generator 33 connected to the first gas source 13 for supplying the first gas 16 (see FIGS. 5 and 6). Operation of the flow generator 33 and controlling pressure of the first gas 16 supplied by the flow generator 33 contributes to regulating the pressure in the inspiratory tube 11 and in turn, the expiratory tube 12. Similarly, the pressure regulation device 22 includes, in part, the second gas source 14 and/or a control valve (not illustrated) that may throttle pressure of the second gas 18 delivered to the inspiratory tube 11 by the second gas source 14. In addition, the pressure regulation device 22 includes a pressure relief valve 61 (see FIG. 3) on the expiratory tube 12 that regulates the pressure in the expiratory tube 12. That is say, venting the exhaled gas 32 and the first gas 16 from the expiratory tube 12 regulates that pressure in the expiratory tube 12 and in turn, in the inspiratory tube 11.

The pressure relief valve 61 may be any suitable device, such as a fixed value positive end expiratory valve (PEEP valve) 62 (see FIG. 3), an adjustable PEEP valve, or a restriction orifice 63 (see FIG. 6). The fixed value PEEP valve 62 may operate on a bias to remain closed until the upstream side of the PEEP valve 62 is exposed to pressure that causes the valve to open, and until the pressure on the upstream side of the PEEP valve 62 falls to, or is below, an operating pressure. An adjustable PEEP valve has a bias that can be adjusted such that the pressure at which the PEEP valve opens can be adjusted. One of the benefits of the breathing circuit 10 disclosed herein is that the pressure at which the exhaled gas 32 and the excess first gas is discharged from the circuit 10, can be controlled by a single pressure regulation device 22, such as the PEEP valve 62, or an orifice (see FIG. 6). This provides a simplified structure and allows for simplified operation. This has been enabled through the interconnection of the inspiratory and the expiratory tubes 11 and 12 for conveying the excess supply of the first gas 16 to the expiratory tube 12.

During patient inhalation and exhalation, the second gas 18 from a second gas source 14 enters the proximal portion 27 of the inspiratory tube 11 via the second gas inlet 17 at a constant rate, and the first gas 16 from a first gas source 13 enters the distal portion 26 via the first gas inlet 15 at a constant rate. At the start of patient exhalation, or during a pause between inhalation ending and exhalation starting, the first non-return valve 19 closes and the second gas 18 back fills the gas passageway of the inspiratory tube 11 in a direction from the second gas inlet 17 toward the distal portion 26. During patient exhalation, the second gas 18 and the first gas 16 form a gas/gas interface that moves along the gas passageway away from the second gas inlet toward the distal portion, thereby storing a volume of the second gas 18 in the inspiratory tube 11 during patient exhalation. The volume of the second gas 18, such as oxygen, that enters the inspiratory tube 11 during exhalation is equal to, or less than, a tidal volume of the patient, thereby minimizing wastage of the second gas 18 by avoiding venting the first gas 16 during exhalation. Ideally, the internal volume of the inspiratory tube 11 is selected such that all of the second gas 18 that is stored in the inspiratory tube 11 and the second gas 18 supplied into the inspiratory tube 11 from the second gas source 14 during patient inhalation is equal to, or less than the tidal volume of the patient. Ideally, the inspiratory tube 11 has a known storage volume of at least two thirds of the patient's tidal volume for storing the second gas.

The patient interface may be a sealed patient interface. For example, the patient interface includes either one or any combination of a full-face mask (also known as an oro-nasal mask), a sealed nasal cannula, a sealed oral mask, a sealed nasal mask, a nasal pillows interface, or a tracheostomy tube.

FIG. 2 is a graph showing a simplified depiction of possible flow rates in the breathing circuit 10 that can be supplied to a patient interface 21 and discharged from the patient interface 21 during inhalation and exhalation over a breathing cycle lasting 1.5 seconds. The dotted line represents the constant supply of the second gas 18 to the breathing circuit 10. In reality, the flow rates will be much more sinusoidal than illustrated in FIG. 2.

Set out below in Table 1 is a list of exemplary internal volumes of an inspiratory tube having a 22 mm internal diameter. For usability and to ensure adequate internal volume is provided, the inspiratory tube has an internal diameter of 22 mm and a length in the ranging from 1.5 m to 1.8 m for adult patients.

TABLE 1

| | Known Tube volume |
|---|---|
| Tube length (m) | (mL) |
| 0.75 | 285 |
| 0.80 | 311 |
| 0.83 | 315 |
| 0.92 | 350 |
| 1.00 | 380 |
| 1.31 | 500 |
| 1.50 | 570 |
| 1.58 | 600 |
| 1.80 | 684 |
| 2.00 | 760 |

The first gas 16 and the second gas 18 can be any suitable breathable gases. For example, the first gas 16 may be any breathable gas such as air, air enriched with oxygen, or any suitable anaesthetic gas. In the situation where the first gas 16 is air, air may be supplied to the inspiratory tube 11 in the range of the 2 to 120 L/min depending on the patient. In the case of an adult patient, air may be supplied to the inspiratory tube 11 in the range of 40 to 120 L/min, or in the range of 50 to 70 L/min. In the case of pediatric patients, air may be supplied to the inspiratory tube 11 in the range of 3 to 50 L/min, or in the range of 4 to 40 L/min. In the case of neonatal patients, air may be supplied to the inspiratory tube 11 in the range of the 2 to 10 L/min, or in the range of 3 to 6 L/min.

Similarly, the second gas 18 may be any breathable gas including any one or any combination of air enriched with oxygen, oxygen, helium, heliox, or any anaesthetic gas. The anaesthetic gas may be nitrous oxide or a 50:50 mixture of nitrous oxide and oxygen gas.

One of the benefits is that the exhaled gas 32 and the first gas 16 are vented from the breathing circuit 10 with little venting, or no venting of the second gas 18. This enables better usage of the second gas 18, such as oxygen in the treatment of patients suffering from respiratory diseases during an outbreak, such as COVID-19. In other words, whilst oxygen efficiency can yield cost savings in treating patients in situations where the supply of oxygen is constrained, an oxygen efficient system will allow more patients to be treated for a given amount of oxygen gas or to allow higher levels of oxygen enrichment to be provided to the same number of patients.

The first and second gases 16, 18 can be supplied by any suitable sources, including pressure cylinders containing the required gases or in-wall hospital supply. In addition, as shown in FIG. 6, the first gas 16 can be supplied by a flow generator 33 such as a blower that is arranged to draw the gas from a storage facility or from ambient air.

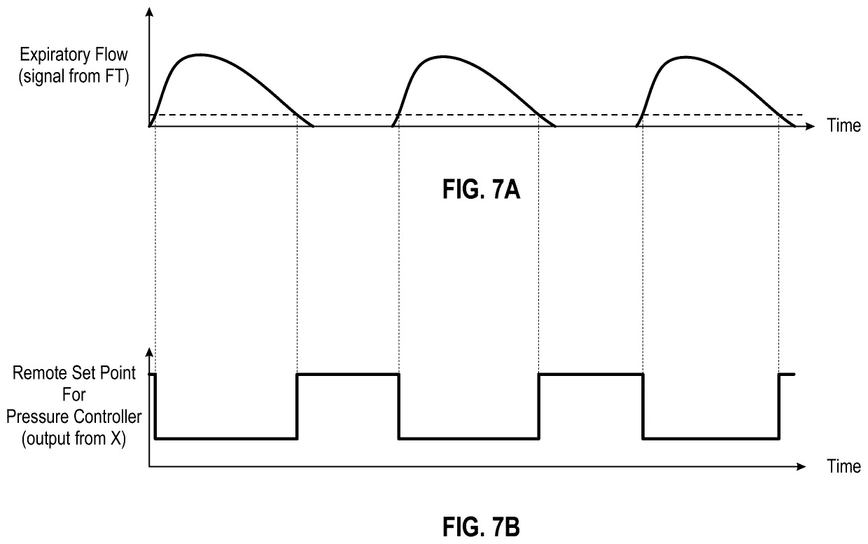

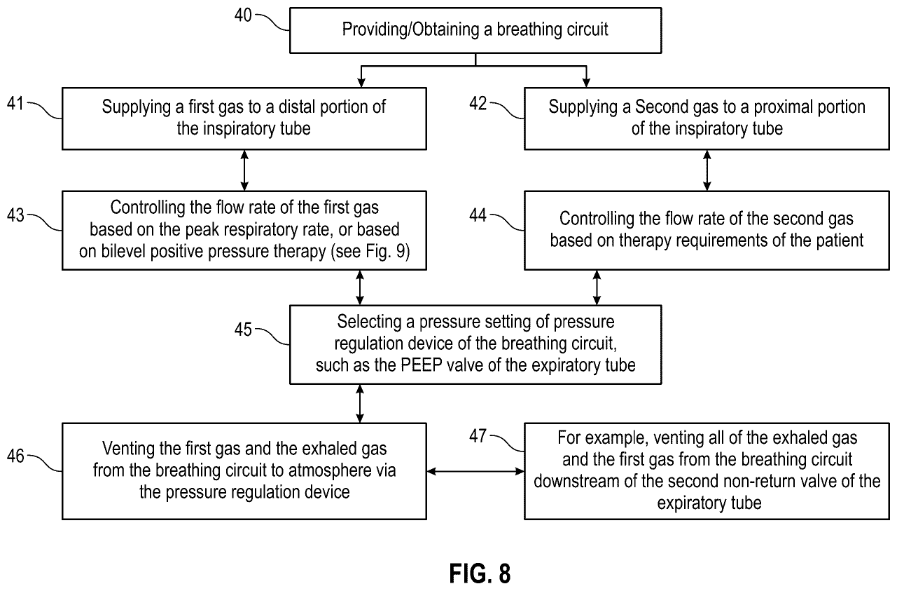

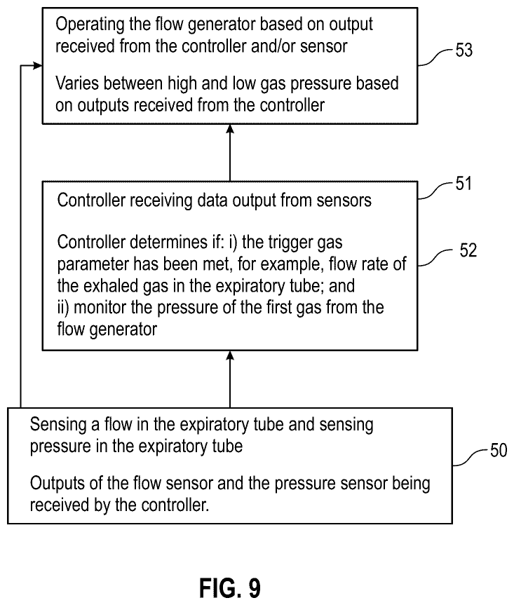

FIGS. 5 and 6 illustrate a breathing circuit 10 that can be operated over a desired pressure range by the pressure regulation device 22 that includes a pressure relief valve 61 (see FIG. 5) or a suitably sized restriction orifice 63 (see FIG. 6). FIG. 8 is a block diagram illustrating the method steps of ventilating a patient using the breathing circuit 10 shown in FIGS. 1, 3, 5 and 6. FIG. 9 is a block diagram illustrating the method steps for operating the flow generator in FIGS. 5 and 6, and corresponds to the graphs shown in FIGS. 7A and 7B.

In the situation where the breathing circuit 10 is used to provide supplemental oxygen gas as the second gas, supplying the second gas/oxygen gas can be adjusted based on patient response, for example the level of oxygen saturation in the patient's blood. Oxygen gas may be supplied at a constant flow rate based on the assessment of the oxygen saturation level of the patient's blood. For example, where the oxygen flow required is between 30 and 50% of the tidal volume, the oxygen flow may be controlled to range from 0.6 to 3.3 L/min.

Based on the assessment of the therapy requirements of the patient, the first gas 16 and the second gas 18 can be supplied to the inspiratory tube 11 at a flow rate that may be determined and controlled.

Controlling 43 the flow rate of the first gas 16 is based on the peak respiratory flow requirement of the patient, and controlling 44 the flow rate of the second gas 18 is based on the fraction of inhaled oxygen gas (FiO2) for the therapy requirement of the patient.

In this situation, the first gas 16 for example air, may be supplied at a constant rate or at a constant pressure. Examples of suitable flow rates range from about 40 to 120 L/min for adult patients, or about 60 L/min. Set out below in Table 2 are examples of flow rates and inspiratory tube 11 volumes for adult patients, pediatric patients and neonatal patients. As can be seen the flow rates and inspiratory tube 11 volumes vary for each category of patient.

TABLE 2

| | | Peak | Example inspiratory | | |
|---|---|---|---|---|---|
| | | inspiratory | tube internal | | Oxygen |
| | | flow | dimensions Dia × | | flow |
| | | (while | length | Air | 30%- |
| | Typical tidal volume | resting) | (volume) | flow | 50% |
| Adult | >300 mL and <700 mL, | 40~60 | 22 mm × 1.5 m | 60 | 0.6~3.3 |
| | typical 500 mL | L/min | (570 mL) | L/min | L/min |
| Paediatric | >50 mL and <300 mL | 4~40 | 15 mm × 1.5 m | 40 | 0.14~2.6 |
| | | L/min | (265 mL) | L/min | L/min |
| Neonatal | >10 mL and <50 mL | 3~6 | 10 mm × 1.5 m | 5 | 0.17~0.6 |
| | | L/min | (118 mL) | L/min | L/min |

In the case of FIGS. 5, 6, 7A, 7B, 8 and 9, supplying 41 the first gas 16 can be varied between a high pressure when the patient is inhaling, and at a lower pressure when the patient is exhaling. In this instance, the breathing circuit 10 includes a sensor 31 that has a pressure sensor 36 and the output 37 of the pressure sensor 36 is used to operate the flow generator 33. Ideally, the flow generator 33 is a variable speed blower and the speed of the blower is variable to ensure that the desired pressure is maintained. The sensor 31 also includes a flow rate sensor 34 in the expiratory tube upstream of the second non-return valve 20. Outputs from the flow sensor 34 and the pressure sensor 36 are used to operate the flow generator 33 between a higher pressure during inhalation and a lower pressure during exhalation. The pressure in the inspiratory and the expiratory tubes 11 and 12 is maintained by adjusting the flow of the first gas 16, such as air, fed to the breathing circuit 10, so that the pressure drop in the gases vented to atmosphere through the restriction orifice is equal to the desired positive pressure of the breathing circuit.

FIG. 5 indicates that the data outputs 35 and 37 of the flow sensor 34 and pressure sensor 36 may be received directly by the flow generator 33 and used to operate the flow generator 33. However ideally, the breathing circuit 10 has a controller 38 that receives the data outputs 35 and 37 of the flow sensor 34 and the pressure sensor 36 that determines when the flow rate of the exhaled gas 32 rises above a trigger level, represented by the dotted line in FIG. 7A. Similarly, the processor of the controller 38 determines when the flow rate of the exhaled gases 32 falls below the trigger level just after the start of the patient exhaling and toward the end of each exhalation cycle.

Depending on the breathing requirements of the patient, including whether the patient is inhaling or exhaling, the controller 38 may, in one example, determine a new desired outlet pressure and adjusts the flow rate or speed of the flow generator 33 to achieve a desired pressure. In another example, the controller may determine a new outlet flow rate or speed of the flow generator 33. The controller 38 outputs a control signal 39 that is calculated from one or more of the sensor outputs 35 and 37 to operate the flow generator 33. The control signal 37 is represented in FIG. 7B as providing a set point pressure for the first gas 16 discharged from the flow generator 33. As can be seen, the set point pressure is lower during exhalation by the patient, and higher during inhalation.

The first gas 16 may be filtered air, ambient air, or ambient air that has been filtered. In the case of FIGS. 5 and 6, the first gas 16 may be pressurized by a flow generator and controlling 44 the flow rate of the second gas 18 is based on the fraction of inhaled oxygen gas (FiO₂) for the therapy requirement of the patient. In the instance where supplemental oxygen therapy is required, the first gas 16, for example air, may be supplied at a rate, in the range of 40 to 120 L/min for adult patients, or approximately 60 L/min. The flow rate of air supplied may exceed the peak respiratory flow rate requirement of the patient. When the breathing circuit includes a flow generator 33 for the first gas 16, FIG. 9 illustrates the steps for controlling the flow rate of the first gas 16. This will be described further below.

The second gas 18, for example oxygen, may be supplied at a flow rate based on the assessment of the oxygen saturation level of the patient's blood. For example, where the oxygen flow required is between 30 and 50% of the tidal volume, the oxygen flow may be controlled to range from 0.6 to 3.3 L/min.

Similarly, the user may select an inspiratory tube 11 having a known internal volume to store the required amount of at least the second gas. For adult patients, where the inspiratory tube 11 has an internal diameter of 22 mm the inspiratory tube 11 may, for example, have a length in the range of 1.5 to 1.8 m. The air flow rates, oxygen gas flow rates and length and internal diameter of the inspiratory tube 11 can be selected by the user. Table 2 above has further examples of flow rates and inspiratory tube 11 volumes for adult patients, pediatric patients and neonatal patients

Supplying 42 the second gas 18, such as oxygen gas to the proximal portion 27 of the inspiratory tube 11, includes the oxygen gas entering the inspiratory tube 11 upstream of the first non-return valve 19. Furthermore the method may include supplying the pressurized air into the distal portion 26 of the inspiratory tube 11 during patient exhalation while a volume of the pressurized oxygen gas enters and is stored in the inspiratory tube 11. As this occurs, excess air supplied to the inspiratory tube 11 is conveyed to the expiratory tube 12. The oxygen gas may be supplied at a pressure greater than the pressure of the air so that the oxygen can backfill the inspiratory tube. In other words, the second gas 18 is supplied at a pressure greater to the inspiratory tube 11 than the first gas 16 so that the second gas 18 can backfill the inspiratory tube 11.

The method may include venting 46, 47 the first gas 16 from the expiratory tube 12 at any stage during the breathing cycle. During exhalation, exhaled gas 32 will also pass the second non-return valve 20 and enter the distal portion of the expiratory tube. The exhaled gas 32 downstream of the second non-return valve 20 is prevented from being re-inhaled and is vented from the circuit 10. In addition, the first gas 16 conveyed to the expiratory tube 12 will be vented from the circuit 10 on account of the second gas 18 entering the inspiratory tube 11 downstream of the first gas 16 entering the inspiratory tube 11.

The first gas 16 can be supplied by a variable flow generator 33 when, for example, the breathing circuit is used to supply bi-level positive air pressure therapy in which the first gas is supplied to the inspiratory tube at an inspiratory pressure that is greater than an expiratory pressure. With reference to FIG. 9, the method steps for controlling the flow generator include detecting the patient's breathing cycle. For example, when the patient exhales. This can be achieved by sensing 50 flow in the expiratory tube 12 by the flow sensor 34 and by sensing pressure in the expiratory tube 12 using the pressure sensor 36. Outputs 35 and 37 of the flow sensor 34 and the pressure sensor 36 may then be received by a controller 51 which has a processor for determining 52 if a trigger parameter has occurred. The dotted line in FIG. 7A intersecting the expiratory flow indicates the trigger has been met shortly after exhalation has started and toward the end of exhalation. The flow generator 33 is then operated 53 based on the output control signal 39 from the controller.

Although not shown in the Figures, the breathing circuit may also include: i) anti-asphyxiation valves, ii) flow rate flags to indicate that the appropriate excess flow necessary to maintain the required positive pressure, iii) purifiers such as anti-viral and bacterial filters for protecting healthcare staff, and iv) humidifiers for humidifying one or more of the first and second gases prior to delivery to the patient to increase patient comfort and reduce dehydration. The humidifiers may be arranged in the inspiratory and the expiratory tubes.

The components of the breathing circuit described herein, including the inspiratory tube and the expiratory tube may be made of any suitable medical grade materials, including flexible plastic tubing that is substantially non-stretchable. Moreover, suitably the inspiratory and the expiratory tubes meet the ISO-5367 standard for compliance.

Conditional language used herein, such as, among others, “can,” “might,” “may,” “for example,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or methods illustrated can be made without departing from the spirit of the disclosure. As will be recognized, certain embodiments of the inventions described herein can be embodied within a form that does not provide all of the features and benefits set forth herein, as some features can be used or practiced separately from others.

Reference Numeral Table

| | |
|---|---|
| breathing circuit | 10 |
| inspiratory tube | 11 |
| expiratory tube | 12 |
| first gas source | 13 |
| second gas source | 14 |
| first gas inlet | 15 |
| first gas | 16 |
| second gas inlet | 17 |
| second gas | 18 |
| first non-return valve | 19 |
| second non-return valve | 20 |
| patient interface | 21 |
| pressure regulation device | 22 |
| bypass tube | 23 |
| distal portion of expiratory tube | 24 |
| proximal portion of expiratory | 25 |
| tube | |
| distal portion of inspiratory tube | 26 |
| proximal portion of inspiratory | 27 |
| tube | |
| flow director/nozzle | 28 |
| narrowing passageway | 29 |
| orifice | 30 |
| sensor | 31 |
| exhaled gas | 32 |
| flow generator | 33 |
| flow sensor | 34 |
| flow sensor output | 35 |
| pressure sensor | 36 |
| pressure sensor output | 37 |
| controller | 38 |
| control signal | 39 |
| providing obtaining breathing circuit | 40 |
| supplying a first gas | 41 |
| supplying a second gas | 42 |
| controlling the flow rate of the first gas | 43 |
| controlling the flow rate of the second gas | 44 |
| selecting the pressure setting of the | 45 |
| pressure regulation device | |
| venting the first gas and exhaled gas | 46 |
| venting all of the exhaled and first gas | 47 |
| sensing a flow parameter in the expiratory | 50 |
| tube | |
| controller receiving an output signal from | 51 |
| the sensor | |
| controller determining if trigger gas | 52 |
| parameter has been met | |
| operating the flow generator based on | 53 |
| output received from the controller and/or | |
| sensor | |
| first supply line | 54 |
| second supply line | 55 |
| device | 56 |
| body | 57 |
| outer wall | 58 |
| bay portion | 59 |
| entrance section | 60 |
| pressure relief valve | 61 |
| PEEP valve | 62 |
| orifice | 63 |

1. A positive pressure breathing circuit for ventilating a patient, the breathing circuit comprising:
an inspiratory tube that is connectable to: i) a patient interface for supplying a breathing gas, ii) a source of a pressurized first gas, and iii) a source of a pressurized second gas, wherein the second gas enters the inspiratory tube downstream to where the first gas enters the inspiratory tube; and
an expiratory tube configured to receive exhaled gas and vent gases including the exhaled gas from the patient interface;
wherein the inspiratory tube is connectable to the expiratory tube so that any excess of the first gas supplied to the inspiratory tube can be conveyed to the expiratory tube, and the expiratory tube is configured so that the excess supply of the first gas conveyed to the expiratory tube and the exhaled gas received by the expiratory tube can be vented from the breathing circuit.

2. The breathing circuit according to claim 1, wherein a distal portion of the inspiratory tube is connectable to the source of the pressurized first gas.

3. The breathing circuit according to claim 1 or 2, wherein a proximal portion of the inspiratory tube is connectable to the source of the pressurized second gas.

4. The breathing circuit according to claim 3, wherein the proximal portion of the inspiratory tube may be connectable to the patient interface.

5. The breathing circuit according to any one of claims 1 to 4, wherein the inspiratory tube includes a first non-return valve.

6. The breathing circuit according to claim 5, wherein the first non-return valve is arranged between the patient interface and the second gas entering the inspiratory tube.

7. The breathing circuit according to claim 5 or 6, wherein the first non-return valve is configured to inhibit the exhaled gas from entering the inspiratory tube.

8. The breathing circuit according to any one of claims 5 to 7, wherein the first non-return valve opens during patient inhalation.

9. The breathing circuit according to any one of claims 5 to 8, wherein the first non-return valve is arranged downstream of where the second gas enters the inspiratory tube.

10. The breathing circuit according to any one of claims 5 to 9, wherein the first non-return valve is located adjacent to the patient interface.

11. The breathing circuit according to any one of claims 5 to 9, wherein the first non-return valve is located proximal to where the second gas enters the inspiratory tube.

12. The breathing circuit according to any one of the preceding claims, wherein the expiratory tube is configured so that all of the excess supply of the first gas conveyed to the expiratory tube and all the exhaled gas in the expiratory tube are vented from the breathing circuit.

13. The breathing circuit according to any one of the preceding claims, wherein the expiratory tube includes a second non-return valve.

14. The breathing circuit according to claim 13, wherein the second non-return valve is configured to inhibit the first gas from entering the patient interface from the expiratory tube.

15. The breathing circuit according to claim 12 or 13, wherein the second non-return valve opens during patient exhalation.

16. The breathing circuit according to any one of claims 13 to 15, wherein the expiratory tube is configured so that the exhaled gas received by the expiratory tube downstream of the second non-return valve is vented from the breathing circuit.

17. The breathing circuit according to any one of claims 13 to 16, wherein the expiratory tube and the inspiratory tube are connectable downstream of the second non-return valve.

18. The breathing circuit according to any one of claims 13 to 17, wherein the expiratory tube is configured so that all of the excess supply of the first gas conveyed to the expiratory tube downstream of the second non-return valve and all the exhaled gas in the expiratory tube downstream of the second non-return valve are vented from the breathing circuit.

19. The breathing circuit according to any one of the preceding claims, wherein the excess supply of the first gas is conveyed from the inspiratory tube to the expiratory tube without passing through the patient interface.

20. The breathing circuit according to any one of the preceding claims, wherein the distal portion of the inspiratory tube is connectable to the distal portion of the expiratory tube for conveying the excess supply of the first gas.

21. The breathing circuit according to claim 20, wherein the proximal portions of the inspiratory and the expiratory tubes are connectable directly or indirectly with the patient interface to form a loop configuration.

22. The breathing circuit according to any one of the preceding claims, wherein the expiratory tube has a substantially constant volume.

23. The breathing circuit according to any one of the preceding claims, wherein the inspiratory tube has a substantially constant volume.

24. The breathing circuit according to any one of the preceding claims, wherein the inspiratory tube is configured so that a volume of the second gas can enter and be loaded in the inspiratory tube whilst the first gas can be supplied to the inspiratory tube, and the first gas supplied in excess can be conveyed to the expiratory tube and vented from the expiratory tube.

25. The breathing circuit according to any one of the preceding claims, wherein the inspiratory tube is configured so that the second gas can enter the inspiratory tube whilst the first gas can be supplied to the inspiratory tube, and the first gas supplied in excess can be conveyed to the expiratory tube and vented from the expiratory tube.

26. The breathing circuit according to any one of the preceding claims, wherein the breathing circuit includes a bypass tube connecting the inspiratory tube and the expiratory tube that conveys the first gas from the inspiratory tube to the expiratory tube.

27. The breathing circuit according to any one of the preceding claims, wherein the inspiratory tube includes a first gas inlet for the first gas, the first gas inlet is configured so that the first gas enters laterally to the inspiratory tube and parallel or coaxial to the bypass tube.

28. The breathing circuit according to claim 27, wherein the first gas inlet includes a first tube connector having multiple limbs, including a first limb that is connectable to a first gas source, a second limb that is connectable to the inspiratory tube, and a third limb that is connectable directly or indirectly to the expiratory tube.

29. The breathing circuit according to claim 28 when appended to claim 23, wherein the third limb is connected to the bypass tube.

30. The breathing circuit according to claim 28, wherein the second limb of the first tube connector is arranged laterally to the first limb, and the third limb is arranged linearly with the first limb, so that the first tube connector provides flow resistance to the first gas entering the inspiratory tube.

31. The breathing circuit according to any one of the preceding claims, wherein the inspiratory tube includes a second gas inlet for the second gas, the second gas inlet being configured so that the second gas enters the inspiratory tube lateral to a longitudinal axis of the inspiratory tube.

32. The breathing circuit according to claim 31, wherein the second gas inlet is arranged downstream of the first non-return valve.

33. The breathing circuit according to claim 31 or 32, wherein the second gas inlet includes a second tube connector having multiple limbs, including a first limb that is connectable to a second gas source, a second limb that is connectable to the inspiratory tube extending toward the first non-return valve, and a third limb that is connectable to the inspiratory tube that extends upstream of the second gas inlet.

34. The breathing circuit according to any one of claims 27 to 33 when appended to claim 26, wherein the breathing circuit includes a third tube connector having multiple limbs, including a first limb that is connectable to the bypass tube, a second limb that is connectable to a distal portion of the expiratory tube downstream of the second non-return valve, and the third limb is connected to the remainder of the distal portion of the expiratory tube extending away from the second non-return valve.

35. The breathing circuit according to claim 34, wherein the third limb is arranged parallel to, or co-axially with the first limb, and the second limb is arranged laterally to the first limb.

36. The breathing circuit according to claim 34 or 35, wherein the arrangement of the first and third limbs minimizes flow resistance for the excess supply of the first gas flowing to the expiratory tube.

37. The breathing circuit according to claim 34, wherein the first limb is arranged parallel to, or co-axially with the second limb, and the third limb is arranged laterally to the first limb.

38. The breathing circuit according to claim 36, wherein the arrangement of the first and second limbs directs the first gas flowing through the third connector toward the second non-return valve to assist in closing the second non-return valve during patient inhalation.

39. The breathing circuit according to any one of the preceding claims when appended to claim 13, further including a flow director for conveying the excess supply of the first gas to the expiratory tube, the flow director has a flow constriction that is configured to increase speed of the first gas passing therethrough and faces toward the second non-return valve so that the gas that exits the flow director assists in biasing the second non-return valve into an operating position.

40. The breathing circuit according to claim 39, wherein the flow constriction includes a nozzle that faces toward the non-return valve.

41. The breathing circuit according to claim 39 or 40, wherein the flow constriction includes a passageway that narrows in a direction of flow of the first gas.

42. The breathing circuit according to any one of claims 39 to 41, wherein the flow constriction includes converging walls in the direction of flow of the first gas.

43. The breathing circuit according to any one of claims 39 to 42, wherein the flow constriction includes an outlet orifice that faces toward the second non-return valve.

44. The breathing circuit according to any one of the preceding claims, wherein the first gas received by the expiratory tube is vented from the breathing circuit without being accumulated or stored, and the expiratory tube is configured so that the exhaled gas passes through the second non-return valve and is vented from the breathing tube without being accumulated or stored.

45. The breathing circuit according to any one of the preceding claims, wherein the expiratory tube is configured so that the first gas and the exhaled gas downstream of the second non-return valve are vented from the breathing circuit without re-entering the inspiratory tube.

46. The breathing circuit according to any one of the preceding claims, wherein the breathing circuit is configured so that there is greater flow resistance for the first gas from the inspiratory tube to the expiratory tube via the patient interface than the flow resistance for the excess of the first gas from the inspiratory tube to the expiratory tube.

47. The breathing circuit according to any one of the preceding claims, further including a pressure regulation device configured to regulate pressure in the expiratory tube.

48. The breathing circuit according to claim 47, wherein the pressure regulation device includes a pressure relief valve configured to vent the first gas and the exhaled gas from the expiratory tube.

49. The breathing circuit according to claim 48, wherein the pressure relief valve is a passive valve, such as an orifice.

50. The breathing circuit according to claim 48, wherein the pressure relief valve is a positive end expiratory pressure valve having a fixed operating pressure or an operating pressure that can be manually adjusted.

51. The breathing circuit according to claim 50, wherein the positive end expiratory pressure valve of the expiratory tube may have a pressure setting ranging from about 2.5 to 20.0 cmH₂O, or ranging from about 8.0 to 12.0 cmH₂O, or about 10.0 cmH₂O.

52. The breathing circuit according to any one of the preceding claims, further including a gas flow generator that supplies the pressurized first gas, and a sensor that senses when the patient breathes, the sensor having an output signal that is used to operate the gas flow generator.

53. The breathing circuit according to claim 52, wherein the sensor includes a flow sensor that measures the flow of the exhaled gas in the expiratory tube and a pressure sensor that measures the pressure of the exhaled gas and the first gas being vented from the expiratory tube.

54. The breathing circuit according to claim 53, wherein the breathing circuit includes a controller that receives the outputs of the flow and pressure sensors, and the controller has a processor that calculates a control output that is used to operate the flow generator and adjust that the gas flow generator to target a desired pressure.

55. The breathing circuit according to any one of the preceding claims, wherein the inspiratory tube has a length ranging from about 0.5 m to 2.5 m, or about a length ranging from 0.75 to 2.0 m, or a length ranging from about 1.5 to 1.8 m.

56. The breathing circuit according to any one of the preceding claims, wherein the inspiratory tube includes plastic tubing having gas passageway of constant diameter.

57. The breathing circuit according to any one of the preceding claims, wherein inspiratory tube has an internal volume ranging from: i) for adult patients about 315 mL to 760 mL, or from about 400 to 600 mL for; ii) for pediatric patients from about 100 mL to 450 mL, or from about 200 to 400 mL; or iii) neonatal patients from about 50 to 200 mL, or from about 100 to 150 mL.

58. The breathing circuit according to any one of the preceding claims, wherein the first gas is pressurized air.

59. The breathing circuit according to any one of the preceding claims, wherein the first gas is pressurized air enriched with oxygen.

60. The breathing circuit according to any one of the preceding claims, wherein the second gas is pressurized oxygen gas.

61. The breathing circuit according to any one of claims 1 to 60, wherein the second gas is a pressurized gas including one or any combination of: oxygen gas, heliox, or an anaesthetic gas.

62. The breathing circuit according to any one of the preceding claims, further including the patient interface.

63. The breathing circuit according to claim 62, wherein the patient interface is a sealed patient interface.

64. A positive pressure breathing circuit for ventilating a patient, the breathing circuit comprising:
an inspiratory tube with a gas passageway that is connectable to: i) a patient interface for supplying a breathing gas, ii) a source of pressurized first gas; and iii) a source of a pressurized second gas, wherein the second gas enters the inspiratory tube downstream to where the first gas enters the inspiratory tube;
an expiratory tube configured to receive exhaled gas and vent gases including the exhaled gases from the patient interface, wherein the inspiratory tube is connectable to the expiratory tube so that any of the first gas supplied to the inspiratory tube supplied in excess flows from the inspiratory tube to the expiratory tube; and
a sensor that senses when the patient breathes, the sensor having an output signal that can be used to operate a gas flow generator for the first gas.

65. The breathing circuit according to claim 64, wherein a distal portion of the inspiratory tube is connectable to the source of the pressurized first gas.

66. The breathing circuit according to claim 64 or 65, wherein a proximal portion of the inspiratory tube is connectable to the source of the pressurized second gas.

67. The breathing circuit according to claim 66, wherein the proximal portion of the inspiratory tube may be connectable to the patient interface.

68. The breathing circuit according to any one of claims 64 to 67, wherein the breathing circuit includes a gas flow generator that supplies the first gas, the gas flow generator receives the output signal from the sensor to operate the gas flow sensor.

69. The breathing circuit according to claim 68, wherein the sensor includes a flow sensor that measures the flow of the exhaled gas in the expiratory tube and a pressure sensor that measures the pressure of the exhaled gas and the first gas being vented from the expiratory tube.

70. The breathing circuit according to claim 69, wherein the breathing circuit includes a controller that receives the outputs of the flow and pressure sensors, and the controller has a processor that calculates a control output that is used to operate the flow generator and adjust the gas flow generator to target a desired pressure.

71. The breathing circuit according to any one of claims 64 to 70, wherein the inspiratory tube includes a first non-return valve.

72. The breathing circuit according to claim 71, wherein the first non-return valve is arranged between the patient interface and the second gas entering the inspiratory tube.

73. The breathing circuit according to claim 71 or 72, wherein the first non-return valve is configured to inhibit the exhaled gas from entering the inspiratory tube.

74. The breathing circuit according to any one of claims 71 to 73, wherein the first non-return valve opens during patient inhalation.

75. The breathing circuit according to any one of claims 71 to 74, wherein the first non-return valve is arranged downstream of where the second gas enters the inspiratory tube.

76. The breathing circuit according to any one of claims 71 to 75 wherein the first non-return valve is located adjacent to the patient interface.

77. The breathing circuit according to any one of claims 71 to 76, wherein the first non-return valve is located proximal to where the second gas enters the inspiratory tube.

78. The breathing circuit according to any one of claims 64 to 77, wherein the expiratory tube is configured so that all of the excess supply of the first gas conveyed to the expiratory tube and all the exhaled gas in the expiratory tube are vented from the breathing circuit.

79. The breathing circuit according to any one of claims 64 to 78, wherein the expiratory tube includes a second non-return valve.

80. The breathing circuit according to claim 79, wherein the second non-return valve is configured to inhibit the first gas from entering the patient interface from the expiratory tube.

81. The breathing circuit according to claim 79 or 80, wherein the second non-return valve opens during patient exhalation.

82. The breathing circuit according to any one of claims 79 to 81, wherein the expiratory tube is configured so that the exhaled gas received by the expiratory tube downstream of the second non-return valve is vented from the breathing circuit.

83. The breathing circuit according to any one of claims 79 to 82, wherein the expiratory tube and the inspiratory tube are connectable downstream of the second non-return valve.

84. The breathing circuit according to any one of claims 79 to 83, wherein the expiratory tube is configured so that all of the excess supply of the first gas conveyed to the expiratory tube downstream of the second non-return valve and all the exhaled gas in the expiratory tube downstream of the second non-return valve are vented from the breathing circuit.

85. The breathing circuit according to any one of claims 64 to 84, wherein the excess supply of the first gas is conveyed from the inspiratory tube to the expiratory tube without passing through the patient interface.

86. The breathing circuit according to any one of claims 64 to 85, wherein the distal portion of the inspiratory tube is connectable to the distal portion of the expiratory tube for conveying the excess supply of the first gas.

87. The breathing circuit according to claim 86, wherein the proximal portions of the inspiratory and the expiratory tubes are connectable directly or indirectly with the patient interface to form a loop configuration.

88. The breathing circuit according to any one of claims 64 to 87, wherein the expiratory tube has a substantially constant volume.

89. The breathing circuit according to any one of claims 64 to 88, wherein the inspiratory tube has a substantially constant volume.

90. The breathing circuit according to any one of claims 64 to 89, wherein the inspiratory tube is configured so that a volume of the second gas can enter and be loaded in the inspiratory tube whilst the first gas can be supplied to the inspiratory tube, and the first gas supplied in excess can be conveyed to the expiratory tube and vented from the expiratory tube.

91. The breathing circuit according to any one of claims 64 to 90, wherein the inspiratory tube is configured so that the second gas can enter the inspiratory tube whilst the first gas can be supplied to the inspiratory tube, and the first gas supplied in excess can be conveyed to the expiratory tube and vented from the expiratory tube.

92. The breathing circuit according to any one of claims 64 to 91, wherein the breathing circuit includes a bypass tube connecting the inspiratory tube and the expiratory tube that conveys the first gas from the inspiratory tube to the expiratory tube.

93. The breathing circuit according to any one of claims 64 to 92, wherein the inspiratory tube includes a first gas inlet for the first gas, the first gas inlet is configured so that the first gas enters laterally to the inspiratory tube and parallel or coaxial to the bypass tube.

94. The breathing circuit according to claim 93, wherein the first gas inlet includes a first tube connector having multiple limbs, including a first limb that is connectable to a first gas source, a second limb that is connectable to the inspiratory tube, and a third limb that is connectable directly or indirectly to the expiratory tube.

95. The breathing circuit according to claim 94 when appended to claim 86, wherein the third limb is connected to the bypass tube.

96. The breathing circuit according to claim 95, wherein the second limb of the first tube connector is arranged laterally to the first limb, and the third limb is arranged linearly with the first limb, so that the first tube connector provides flow resistance to the first gas entering the inspiratory tube.

97. The breathing circuit according to any one of claims 64 to 96, wherein the inspiratory tube includes a second gas inlet for the second gas, the second gas inlet being configured so that the second gas enters the inspiratory tube lateral to a longitudinal axis of the inspiratory tube.

98. The breathing circuit according to claim 97, wherein the second gas inlet is arranged downstream of the first non-return valve.

99. The breathing circuit according to claim 97 or 98, wherein the second gas inlet includes a second tube connector having multiple limbs, including a first limb that is connectable to a second gas source, a second limb that is connectable to the inspiratory tube extending toward the first non-return valve, and a third limb that is connectable to the inspiratory tube that extends upstream of the second gas inlet.

100. The breathing circuit according to any one of claims 96 to 99 when appended to claim 92, wherein breathing circuit includes a third tube connector having multiple limbs, including a first limb that is connectable to the bypass tube, a second limb that is connectable to a distal portion of the expiratory tube downstream of the second non-return valve, and the third limb is connected to the remainder of the distal portion of the expiratory tube extending away from the second non-return valve.

101. The breathing circuit according to claim 100, wherein the third limb is arranged parallel to, or co-axially with the first limb, and the second limb is arranged laterally to the first limb.

102. The breathing circuit according to claim 100 or 101, wherein the arrangement of the first and third limbs minimizes flow resistance for the excess supply of the first gas flowing to the expiratory tube.

103. The breathing circuit according to claim 100, wherein the first limb is arranged parallel to, or co-axially with the second limb, and the third limb is arranged laterally to the first limb.

104. The breathing circuit according to claim 101, wherein the arrangement of the first and second limbs directs the first gas flowing through the third connector toward the second non-return valve to assist in closing the second non-return valve during patient inhalation.

105. The breathing circuit according to any one of claims 64 to 104 when appended to claim 79, further including a flow director for conveying the excess supply of the first gas to the expiratory tube, the flow director has a flow constriction that is configured to increase speed of the first gas passing therethrough and faces toward the second non-return valve so that the gas that exits the flow director assists to bias the second non-return valve into an operating position.

106. The breathing circuit according to claim 105, wherein the flow constriction includes a nozzle that faces toward the non-return valve.

107. The breathing circuit according to claim 105 or 106, wherein the flow constriction includes a passageway that narrows in a direction of flow of the first gas.

108. The breathing circuit according to any one of claims 105 to 107, wherein the flow constriction includes converging walls in the direction of flow of the first gas.

109. The breathing circuit according to any one of claims 105 to 108, wherein the flow constriction includes an outlet orifice that faces toward the second non-return valve.

110. The breathing circuit according to any one of claims 64 to 109, wherein the first gas received by the expiratory tube is vented from the breathing circuit without being accumulated or stored, and the expiratory tube is configured so that the exhaled gas passes through the second non-return valve and is vented from the breathing tube without being accumulated or stored.

111. The breathing circuit according to any one of claims 64 to 110, wherein the expiratory tube is configured so that the first gas and the exhaled gas downstream of the second non-return valve are vented from the breathing circuit without re-entering the inspiratory tube.

112. The breathing circuit according to any one of claims 64 to 111, wherein the breathing circuit is configured so that there is greater flow resistance for the first gas from the inspiratory tube to the expiratory tube via the patient interface than the flow resistance for the excess of the first gas from the inspiratory tube to the expiratory tube.

113. The breathing circuit according to any one of claims 64 to 112, further including a pressure regulation device configured to regulate pressure in the expiratory tube.

114. The breathing circuit according to claim 113, wherein the pressure regulation device includes a pressure relief valve configured to vent the first gas and the exhaled gas from the expiratory tube.

115. The breathing circuit according to claim 114, wherein the pressure relief valve is a passive valve, such as an orifice.

116. The breathing circuit according to claim 114, wherein the pressure relief valve is a positive end expiratory pressure valve having a fixed operating pressure or an operating pressure that can be manually adjusted.

117. The breathing circuit according to claim 116, wherein the positive end expiratory pressure valve of the expiratory tube may have a pressure setting ranging from about 2.5 to 20.0 cmH₂O, or ranging from about 8.0 to 12.0 cmH₂O, or about 10.0 cmH₂O.

118. The breathing circuit according to any one of claims 64 to 117, wherein the inspiratory tube has a length ranging from about 0.5 m to 2.5 m, or about a length ranging from 0.75 to 2.0 m, or a length ranging from about 1.5 to 1.8 m.

119. The breathing circuit according to any one of claims 64 to 118, wherein the inspiratory tube includes plastic tubing having gas passageway of constant diameter.

120. The breathing circuit according to any one of claims 64 to 119, wherein inspiratory tube has an internal volume ranging from: i) for adult patients about 315 ml to 760 mL, or from about 400 to 600 mL for; ii) for pediatric patients from about 100 mL to 450 mL, or from about 200 to 400 mL; or iii) neonatal patients from about 50 to 200 mL, or from about 100 to 150 mL.

121. The breathing circuit according to any one of claims 64 to 120, wherein the first gas is pressurized air.

122. The breathing circuit according to any one of claims 64 to 121, wherein the first gas is pressurized air enriched with oxygen.

123. The breathing circuit according to any one of claims 64 to 122, wherein the second gas is pressurized oxygen gas.

124. The breathing circuit according to any one of claims 64 to 122, wherein the second gas is a pressurized gas including one or any combination of: oxygen gas, heliox, or an anaesthetic gas.

125. The breathing circuit according to any one of claims 64 to 124, further including the patient interface.

126. The breathing circuit according to claim 125, wherein the patient interface is a sealed patient interface.

127. A device that can be arranged between a gas non-return valve and a gas tube, the device includes a body having a bay portion that connects to the non-return valve, and a flow director extending from the bay portion that receives gas from the gas tube, wherein the flow director has a flow constriction that is configured to increase speed of the gas passing therethrough and faces toward the non-return valve so that the gas that exits the flow director assists in biasing the non-return valve into an operating position.

128. The device according to claim 127, wherein the bay portion can be fixedly connected to an outlet of the non-return valve.

129. The device according to claim 127, wherein the bay portion can be removably connected to an outlet of the non-return valve.

130. The device according to claim 127, wherein the bay portion can be fixedly connected to the gas tube.

131. The device according to claim 127, wherein the bay portion can be removably connected to the gas tube.

132. The device according to any one of claims 127 to 131, wherein the operating position of the non-return valve is a closed position.

133. The device according to any one of claims 127 to 132, wherein the body has an outer wall having opposite ends that connect to the non-return valve and the gas tube, the outer wall also defining the bay portion as a cavity between the opposite ends.

134. The device according to any one of claims 127 to 133, wherein the flow constriction has a nozzle that faces toward the non-return valve.

135. The device according to any one of claims 127 to 134, wherein the flow constriction includes a converging portion that narrows in a direction of flow of the first gas.

136. The device according to any one of claims 127 to 135, wherein the flow constriction has a discharge portion having an outlet for discharging the gas passing through the flow director, in which the discharge portion has a constant diameter.

137. A method of ventilating a patient, the method including steps of:
a) providing a positive pressure breathing circuit including:
an inspiratory tube that is connectable to: i) a patient interface, ii) a source of pressurized first gas, and iii) a source of a pressurized second gas, wherein the second gas enters the inspiratory tube downstream to where the first gas enters the inspiratory tube;
an expiratory tube configured to receive exhaled gas and vent gases including the exhaled gases from the patient interface;
wherein the inspiratory tube is connectable to the expiratory tube; and
b) supplying a pressurized second gas into the inspiratory tube; and
c) supplying the pressurized first gas into the inspiratory tube, and during patient exhalation a volume of the pressurized second gas that enters and is stored in the inspiratory tube displaces the first gas from the inspiratory tube and is conveyed to the expiratory tube.

138. The method according to claim 137, wherein the step of providing the breathing circuit includes a distal portion of the inspiratory tube being connectable to the source of the pressurized first gas.

139. The method according to claim 137, wherein the step of providing the breathing circuit includes a proximal portion of the inspiratory tube being connectable to the source of the pressurized second gas.

140. The method according to claim 137, wherein the step of providing the breathing circuit includes the proximal portion of the inspiratory tube being connectable to the patient interface.

141. The method according to any one of claims 137 to 140, wherein the step of supplying the first gas to the inspiratory tube includes supplying in an excess amount and conveying excess supply of the first gas from the inspiratory tube to the expiratory tube, and venting the excess supply of the first gas and the exhaled gas from the expiratory tube.

142. The method according to claim 141, wherein the step of providing the breathing circuit includes the inspiratory tube may include a first non-return valve.

143. The method according to claim 142, wherein the first non-return valve inhibits the exhaled gas from entering the inspiratory tube.

144. The method according to any one of claims 137 to 143, wherein the step of providing the breathing circuit includes the expiratory tube may include a second non-return valve.

145. The method according to claim 144, wherein the second non-return valve inhibits the first gas from entering the patient interface from the expiratory tube.

146. The method according to any one of claims 137 to 145 when appended to claim 142, wherein the step of supplying the pressurized second gas into the proximal portion of the inspiratory tube may include the second gas entering the inspiratory tube upstream from the first non-return valve.

147. The method according to claim 144 or 145, wherein the first gas and the exhaled gas is vented from the breathing circuit downstream of the second non-return valve.

148. The method according to any one of claims 141 to 147, wherein the step of supplying the pressurized first gas may be carried out continuously to the distal portion of the inspiratory tube.

149. The method according to any one of claims 141 to 148, wherein the step of supplying the pressurized first gas may be carried out at a rate that is greater than or equal to peak inspiratory flow rate of a patient.

150. The method according to any one of claims 141 to 149, wherein the step of providing the breathing circuit includes the breathing circuit having a pressure regulating device, and the method includes regulating the pressure in the breathing circuit.

151. The method according to claim 150, wherein the pressure regulating device includes a positive end expiratory valve (PEEP valve) in the expiratory tube downstream of the second non-return valve, and the method includes operating the PEEP valve to vent the exhaled gas and the first gas from the breathing circuit at a desired pressure.

152. The method according to claim 151, wherein the method includes selecting a pressure setting of the PEEP valve of the expiratory tube within a range from about 2.5 to 20.0 cmH₂O, or a range from about 8.0 to 12.0 cmH₂O, or about 10.0 cmH₂O.

153. The method according to any one of claims 141 to 152, wherein the breathing circuit provided includes a gas flow sensor in the expiratory tube upstream of the second non-return valve and a pressure sensor located downstream of the second non-return valve, and the method includes detecting when the patient is exhaling based on an output of the gas flow sensor and detecting the pressure of first gas being supplied to the breathing circuit based on an output of the pressure sensor.

154. The method according to claim 153, wherein the breathing circuit provided includes a controller that receives the outputs of the gas flow and pressure sensors, and the controller has a processor that calculates a control output that is used to operate the flow generator and adjust the flow generator to target a desired pressure.

155. The method according to any one of claims 141 to 154, wherein during patient exhalation, the second gas entering the inspiratory tube can flow backwards along the inspiratory tube which acts as a constant pressure storage volume by displacing air out of the inspiratory tube via the further positive end expiratory pressure valve of the inspiratory tube.

156. The method according to any one of claims 141 to 155, wherein during patient inhalation, the breathing gas from the inspiratory tube will initially be the second gas that had been stored in the inspiratory tube and then the first gas.

157. The method according to any one of claims 141 to 156, wherein the first gas is air and is supplied to the inspiratory tube in the ranges: i) for adults patients from about 40 to 120 L/min, or about 50 to 70 L/min, ii) for pediatric patients from about 3 to 50 L/min, or from about 4 to 40 L/min, or iii) for neonatal patients from about 2 to 10 L/min, or at a range from about 3 to 6 L/min.

158. The method according to any one of claims 141 to 157, wherein the inspiratory tube of the breathing circuit provided has a length ranging from about 0.5 m to 2.5 m, or a length ranging from about 0.75 to 2.0 m, or a length ranging from about 1.5 to 1.8 m.

159. The method according to any one of claims 141 to 157, wherein the inspiratory tube of the breathing circuit provided has a gas passage of constant diameter, in which the diameter may range from about 18 to 25 mm, or a diameter about 22 mm.

160. The method according to any one of claims 141 to 159, wherein the inspiratory tube of the breathing circuit provided has an internal volume ranging from about 100 ml to 760 mL, for storing the second gas and some of the first gas.

161. The method according to claim 160, wherein the internal volume of the inspiratory tube ranges: i) for adult patient from about 315 mL to 760 mL, or from about 400 to 600 mL, ii) for pediatric patients from about 100 mL to 450 mL, or from about 200 to 400 mL, and iii) for neonatal patients from about 50 to 200 mL, or range from about 100 to 150 mL.

162. The method according to any one of claims 141 to 161, wherein the first gas is pressurized air.

163. The method according to any one of claims 141 to 161, wherein the first gas is pressurized air enriched with oxygen.

164. The method according to any one of claims 141 to 161, wherein the second gas is pressurized oxygen gas.

165. The method according to any one of claims 141 to 164, wherein the second gas is a pressurized gas including one or any combination of: oxygen gas, heliox, or an anaesthetic gas.
